(19) United States
(12) Patent Application Publication
(10) Pub. No.: US 2024/0358873 A1
(43) Pub. Date: Oct. 31, 2024

(54) Method of Inactivating Pathogens, Control System and Robot System

(71) Applicant: ABB Schweiz AG, Baden (CH)

(72) Inventors: Pietro Falco, Parete (CE) (IT); Jordi Artigas, Barcelona (ES)

(21) Appl. No.: 18/577,557

(22) PCT Filed: Jul. 20, 2021
(86) PCT No.: PCT/EP2021/070287

§ 371 Date: Jan. 8, 2024

(51) Int. Cl. A61L 2/24; A61L 2/26

(52) U.S. Cl. CPC A61L 2/24; A61L 2/26; A61L 2202/14; A61L 2202/16; A61L 2202/25

(57) ABSTRACT

A method of inactivating pathogens in an environment, the method including moving a base of a mobile robot carrying a pathogen detector on a base surface; moving the pathogen detector by means of a manipulator; obtaining pathogen data by means of the pathogen detector, the pathogen data being indicative of a presence of pathogens in one or more detection regions of the environment; moving a base of a mobile robot carrying an inactivation device on the base surface based on the pathogen data; moving the inactivation device based on the pathogen data by means of the manipulator carrying the inactivation device; and controlling the inactivation device to inactivate the pathogens.

### TECHNICAL FIELD

The present disclosure generally relates to inactivation of pathogens in an environment. In particular, a method of inactivating pathogens in an environment by means of a robot system, a control system for controlling inactivation of pathogens in an environment by means of a robot system, and a robot system, are provided.

### BACKGROUND

Many environments pose a risk of humans being exposed to infectious pathogens. Examples of such environments include hospitals, public areas, retail houses, warehouses and factories. Disinfection of surfaces in such environments is often important to reduce risks of infections. Most disinfection procedures are today carried out more or less manually. Thus, the level of automation is low. Disinfection procedures involving manual labor are costly and time-consuming.

CN 109701060 A discloses a disinfection control method using a portable disinfecting robot. The method comprises controlling the disinfecting robot to move along a preset disinfection road, performing real-time acquisition on the quantity of bacteria, and obtaining a corresponding disinfection period from a corresponding table.

### SUMMARY

One object of the present disclosure is to provide an efficient method of inactivating pathogens in an environment by means of a robot system.

A further object of the present disclosure is to provide a method of inactivating pathogens in an environment by means of a robot system, which method can rapidly inactivate pathogens.

A still further object of the present disclosure is to provide a method of inactivating pathogens in an environment by means of a robot system, which method enables a reliable inactivation of pathogens.

A still further object of the present disclosure is to provide a method of inactivating pathogens in an environment by means of a robot system, which method enables human presence.

A further object of the present disclosure is to provide a method of inactivating pathogens in an environment by means of a robot system, which method solves several or all of the foregoing objects in combination.

A still further object of the present disclosure is to provide a control system for controlling inactivation of pathogens in an environment by means of a robot system, which control system solves one, several or all of the foregoing objects.

A still further object of the present disclosure is to provide a robot system solving one, several or all of the foregoing objects.

According to a first aspect, there is provided a method of inactivating pathogens in an environment by means of a robot system, the robot system comprising at least one mobile robot, each mobile robot comprising a base, a traction arrangement configured to move the base on a base surface of the environment, and at least one manipulator connected to the base and movable relative to the base; a pathogen detector carried by one of the at least one manipulator, the pathogen detector being configured to detect the presence of pathogens in a detection region; and an inactivation device carried by one of the at least one manipulator, the inactivation device being configured to inactivate pathogens; the method comprising moving the base on the base surface; moving the pathogen detector by means of the manipulator; obtaining pathogen data by means of the pathogen detector, the pathogen data being indicative of a presence of pathogens in one or more detection regions of the environment; moving the base on the base surface based on the pathogen data; moving the inactivation device based on the pathogen data by means of the manipulator; and controlling the inactivation device to inactivate the pathogens.

By controlling the inactivation device to move based on the pathogen data, the inactivation device can accurately approach areas and inactivate the detected pathogens. Moreover, by controlling the inactivation device based on the pathogen data, in contrast to controlling the inactivation device over the entire environment, the method enables performance of only local inactivations, when needed. Thus, according to one variant, the method may comprise controlling the inactivation device to act only on regions where pathogens have been detected by the pathogen detector. The method thereby enables an efficient inactivation of pathogens in a short time.

Furthermore, the provision of each of the pathogen detector and the inactivation device on a manipulator of a mobile robot makes detections and inactivations of pathogens more accurate. Due to the one or more manipulators, the robot system also enables agile, dexterous and accurate movements of the pathogen detector and the inactivation device. The inactivation performance of the method is thereby improved.

The pathogen detector can be moved rapidly in three dimensions and can reach pathogens in areas that are difficult to reach by humans. Each manipulator may be a serial manipulator comprising three or more axes, such as six or seven axes.

The pathogen data may indicate a presence of pathogens in the environment. Optionally, the pathogen data may also indicate a type of pathogen. The pathogen detector may however be configured to detect only the presence, and an optional amount of, one or a few types of pathogens and communicate this information as pathogen data.

The pathogen data may optionally also contain position information of the pathogens. Thus, the pathogen detector may communicate pathogen data containing such position information. If such position information is not provided by the pathogen detector, the mobile robot carrying the pathogen detector may determine the position information and add this to the pathogen data. In this case, the position information may be determined by the mobile robot based on a position of the manipulator when the pathogen data is obtained by the pathogen detector.

The pathogen detector may be a biosensor, such as an optoacoustic biosensor or an optical biosensor. An optical biosensor may for example combine an optical effect and a thermal effect to reliably detect pathogens.

The inactivation device may be configured to expose the pathogens to radiation and/or to eject an inactivation media onto the pathogens. According to one example, the inactivation device comprises an ultraviolet (UV) emitter, such as an UVC emitter having wavelengths between 200 nm and 280 nm. The UV emitter may be of low intensity enabling human proximity. According to a further example, the inactivation device is configured to eject a chemical disinfectant.

The moving of the base on the base surface may comprise moving the base carrying the pathogen detector. The moving of the pathogen detector by means of the manipulator may comprise moving the manipulator carrying the pathogen detector. The moving of the base on the base surface based on the pathogen data may comprise moving the base carrying the inactivation device. The moving of the inactivation device based on the pathogen data by means of the manipulator may comprise moving the manipulator carrying the inactivation device.

Throughout the present disclosure, the inactivation of pathogens may be a disinfection of pathogens. The detection regions may for example contain various surfaces, edges and contours within the environment. The base surface may be a floor.

The traction arrangement may comprise a plurality of wheels. The wheels may drive a base in the form of a platform. At least one of the wheels may be a driving wheel and at least one of the wheels may be a steering wheel. The wheels may provide two or three degrees of freedom of the base on the base surface.

The method may comprise moving the base carrying the pathogen detector simultaneously with moving the pathogen detector by means of the manipulator carrying the pathogen detector and/or simultaneously with obtaining pathogen data by means of the pathogen detector. Alternatively, or in addition, the method may comprise moving the pathogen detector by means of the manipulator carrying the pathogen detector simultaneously with obtaining pathogen data by means of the pathogen detector.

The method may comprise moving the base carrying the inactivation device simultaneously with moving the inactivation device by means of the manipulator carrying the inactivation device and/or simultaneously with controlling the inactivation device to inactivate the pathogens. Alternatively, or in addition, the method may comprise moving the inactivation device by means of the manipulator carrying the inactivation device simultaneously with controlling the inactivation device to inactivate the pathogens.

Each base and each manipulator may be moved autonomously. The method thereby enables an autonomous detection and inactivation of pathogens. The base and the manipulator carrying the inactivation device may be moved autonomously based on the pathogen data. The entire method may be carried out automatically enabling humans to not be involved in the inactivation process.

The method may further comprise providing a map representing the environment; and mapping the pathogen data in the map. The method may thus comprise an automatic sensing and mapping of a distribution of pathogens in the environment and an automatic inactivation of the pathogens. The map may be a grid-based map. In this case, a pathogen density value, determined based on the pathogen data, may be associated with one, several or all cells of the grid-based map.

The map representing the environment may be provided by a mobile robot carrying a navigation sensor, such as the mobile robot carrying the pathogen detector and/or the inactivation device. Examples of navigation sensors include one or more cameras (such as RGB-D cameras), a radar and a lidar device. Alternatively, the map representing the environment may be provided in other ways, such as from a CAD (computer-aided design) model.

The moving and controlling of the inactivation device may be made based on the map.

The method may further comprise providing a detection strategy based on the map. In this case, the moving of the pathogen detector may be performed based on the detection strategy. The detection strategy may make sure that every part of surfaces in the environment is scanned by the pathogen detector.

The base and the manipulator carrying the pathogen detector may be moved in accordance with the detection strategy. Initially, the detection strategy may be such that the pathogen detector scans all regions within an environment. The detection strategy for the pathogen detector may then be updated based on the pathogen data. For example, regions where pathogens have been detected, and/or have frequently been detected, may be prioritized in subsequent scans by the pathogen detector.

The method may further comprise providing an inactivation strategy based on the map. In this case, the moving and controlling of the inactivation device may be performed based on the inactivation strategy.

The detection strategy and/or the inactivation strategy may be improved by one or more machine learning algorithms. In this way, the machine learning algorithm can learn locations and optionally types of frequently occurring pathogens in the environment, and further improve the inactivation strategy. For example, one machine learning algorithm may build a model of the detection strategy using the pathogen data as training data. A further machine learning algorithm may build a model of the inactivation strategy using the pathogen data and one or more previous inactivation strategies as training data.

The detection strategy and the inactivation strategy may be alternatingly executed by the robot system. As an alternative, several runs of the inactivation strategy may be executed between two runs of the detection strategy. For example, in some environments, pathogens appear more or less at the same location every time. If the environment contains such locations prone to accumulate pathogen presence, the inactivation strategy may be executed more often than the detection strategy, at least for those locations.

The map may be three-dimensional. By mapping the pathogen data in a three-dimensional map and controlling the inactivation device by means of a manipulator of a mobile robot, the inactivation strategy can be made more efficient. Moreover, the inactivation device can be efficiently moved to access regions for deactivation of pathogens, such as hidden areas or areas at large heights.

The method may further comprise issuing an alarm if the pathogen data indicates a level of pathogens in a detection region above a threshold value. The alarm may be audible and/or visual. In this way, humans in the environment can be commanded to exit the environment and/or a process may be stopped.

One of the at least one manipulator may comprise an end effector. That is, an end effector in addition to the pathogen detector and/or the inactivation device. Thus, one or each manipulator may be used also for carrying out different tasks and does not have to be limited to detection and/or inactivation of pathogens. One example of an additional tasks is transportation of items. One example of an end effector is a gripper.

Each mobile robot may be a collaborative robot. The method can thereby be carried out in human populated environment without needing to interrupt any process carried out by nearby humans.

Throughout the present disclosure, a collaborative robot may alternatively be referred to as a human-collaborative robot configured to be operated while sharing a workspace with a human. The collaborative robot can operate in proximity to a human operator without the need for any safety arrangements.

The collaborative robot according to the present disclosure may be a truly collaborative robot, i.e. a collaborative robot that is constructed to not be capable of injuring humans. The truly collaborative robot may have a mass of 100 kg or less. Alternatively, or in addition, the truly collaborative robot may comprise one or more manipulators driven at a power that is less than 80 W. A truly collaborative robot differs from an originally non-collaborative industrial robot that is retrofitted with sensor to be made collaborative. One example of a truly collaborative robot is the YuMi® by ABB.

The pathogen detector and the inactivation device may be noninvasive. Examples of noninvasive pathogen detectors include optoacoustic biosensors and optical biosensors. Examples of noninvasive inactivation devices include radiation emitting devices, such as UV emitters.

The pathogen detector and the inactivation device may be provided on a common manipulator. In this way, the method is made cost-efficient and claims a smaller area for operation. Alternatively, the robot system may comprise a first mobile robot comprising a first manipulator carrying the pathogen detector and a second mobile robot comprising a second manipulator carrying the inactivation device.

According to a second aspect, there is provided a control system for controlling inactivation of pathogens in an environment by means of a robot system, the robot system comprising at least one mobile robot, each mobile robot comprising a base, a traction arrangement configured to move the base on a base surface of the environment, and at least one manipulator connected to the base and movable relative to the base; a pathogen detector carried by one of the at least one manipulator, the pathogen detector being configured to detect the presence of pathogens in a detection region; and an inactivation device carried by one of the at least one manipulator, the inactivation device being configured to inactivate pathogens; the control system comprising at least one data processing device and at least one memory having at least one computer program stored thereon, the at least one computer program comprising program code which, when executed by the at least one data processing device, causes the at least one data processing device to perform the steps of commanding movement of the base carrying the pathogen detector on the base surface; commanding movement of the pathogen detector by means of the manipulator carrying the pathogen detector; receiving pathogen data from the pathogen detector, the pathogen data being indicative of a presence of pathogens in one or more detection regions of the environment; commanding movement of the base carrying the inactivation device on the base surface based on the pathogen data; commanding movement of the inactivation device based on the pathogen data by means of the manipulator carrying the inactivation device; and controlling the inactivation device to inactivate the pathogens.

The control system may control a robot system of any type according to the first aspect.

The at least one computer program may comprise program code which, when executed by the at least one data processing device, causes the at least one data processing device to perform, or command performance of, the method according to any variant of the first aspect or any variant according to remainder of the present disclosure.

The at least one computer program may comprise program code which, when executed by the at least one data processing device, causes the at least one data processing device to control each base and each manipulator to move autonomously.

The at least one computer program may comprise program code which, when executed by the at least one data processing device, causes the at least one data processing device to provide a map representing the environment, and mapping the pathogen data in the map.

The at least one computer program may comprise program code which, when executed by the at least one data processing device, causes the at least one data processing device to control movements of the manipulator carrying the inactivation device and control the inactivation device based on the map.

The at least one computer program may comprise program code which, when executed by the at least one data processing device, causes the at least one data processing device to provide a detection strategy based on the map, and to control movement of the manipulator carrying the pathogen detector based on the detection strategy.

The at least one computer program may comprise program code which, when executed by the at least one data processing device, causes the at least one data processing device to provide an inactivation strategy based on the map, control movements of the manipulator carrying the inactivation device, and control the inactivation device based on the inactivation strategy. The map may be three-dimensional.

The at least one computer program may comprise program code which, when executed by the at least one data processing device, causes the at least one data processing device to command issuance of an alarm if the pathogen data indicates a level of pathogens in a detection region above a threshold value.

One of the at least one manipulator may comprise an end effector. Each mobile robot may be a collaborative robot. The pathogen detector and the inactivation device may be noninvasive. The pathogen detector and the inactivation device may be provided on a common manipulator.

According to a third aspect, there is provided a robot system comprising the at least one mobile robot; the pathogen detector; the inactivation device; and a control system according to the second aspect. The robot system may be of any type as described in connection with the first and second aspects.

### BRIEF DESCRIPTION OF THE DRAWINGS

Further details, advantages and aspects of the present disclosure will become apparent from the following description taken in conjunction with the drawings, wherein:

FIG. 1: schematically represents a side view of a robot system comprising a mobile robot;

FIG. 2: schematically represents components of the mobile robot;

FIG. 3: schematically represents the mobile robot in an environment containing pathogens; and

FIG. 4: schematically represents a side view of a further example of a robot system comprising two mobile robots.

### DETAILED DESCRIPTION

In the following, a method of inactivating pathogens in an environment by means of a robot system, a control system for controlling inactivation of pathogens in an environment by means of a robot system, and a robot system, will be described. The same or similar reference numerals will be used to denote the same or similar structural features.

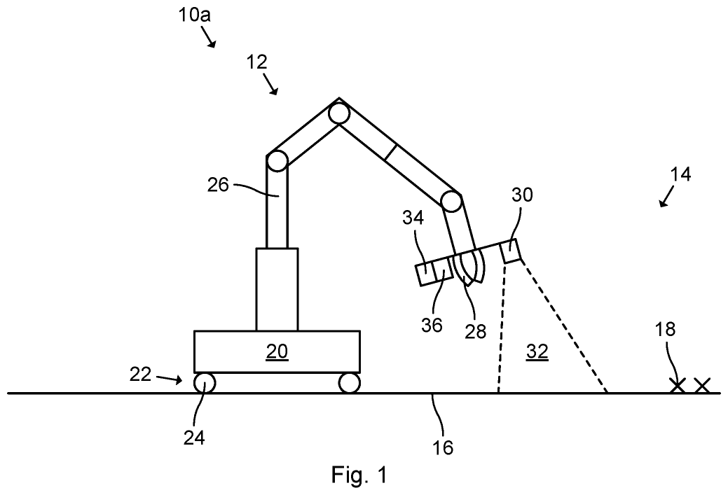

FIG. 1 schematically represents a side view of a robot system 10a. The robot system 10a of this example comprises a mobile robot 12. The robot system 10a of this example comprises only the mobile robot 12. Thus, any reference to the robot system 10a applies equally to the mobile robot 12, and vice versa, in this example.

The mobile robot 12 is positioned in an environment 14 comprising a base surface 16, here exemplified as a floor. The mobile robot 12 of this example is a collaborative robot designed to operate in close proximity with humans. FIG. 1 schematically illustrates pathogens 18 on the floor, represented with crosses. The pathogens 18 may for example be virus or bacteria.

The mobile robot 12 of this example comprises a base 20 and a traction arrangement 22 for moving the base 20 over the base surface 16. The base 20 is here exemplified as a platform having a generally horizontal main extension. The traction arrangement 22 is here exemplified as a plurality of wheels 24 (here four), among which at least one wheel 24 may be a drivable wheel and at least one wheel 24 may be a steerable wheel.

The mobile robot 12 further comprises a manipulator 26 connected to the base 20. The manipulator 26 of this example is a serial manipulator movable in six axes relative to the base 20. The manipulator 26 comprises an end effector, here exemplified as a gripper 28. By means of the gripper 28, the manipulator 26 can perform various tasks, such as transportation and handling tasks.

The mobile robot 12 further comprises a pathogen detector 30. The pathogen detector 30 is carried by the manipulator 26. The pathogen detector 30 is here fixed to a most distal link of the manipulator 26. The pathogen detector 30 thereby has a known position offset from a tool center point (TCP) of the manipulator 26.

The pathogen detector 30 is configured to detect the presence of pathogens 18 in a detection region 32. The detection region 32 is shown in FIG. 1 as a region in front of the pathogen detector 30. The pathogen detector 30 is here exemplified as a noninvasive biosensor. The pathogen detector 30 may for example be a dual-functional localized surface plasmon resonance (LSPR) biosensor.

The mobile robot 12 further comprises an inactivation device 34. The inactivation device 34 is carried by the manipulator 26. Similarly to the pathogen detector 30, the inactivation device 34 is here fixed to the most distal link of the manipulator 26. Also the inactivation device 34 thereby has a known position offset from the TCP of the manipulator 26.

The inactivation device 34 is configured to disinfect pathogens 18. The inactivation device 34 is here exemplified as an UVC emitter configured to emit radiation having wavelengths between 200 nm and 280 nm, which are not harmful to humans.

The mobile robot 12 of this example further comprises a navigation sensor 36. Also the navigation sensor 36 is carried by the manipulator 26 and is fixed to the most distal link thereof. The navigation sensor 36 is here exemplified as a lidar sensor. By means of the navigation sensor 36, the mobile robot 12 can provide a three-dimensional map of the environment 14. The map may be used for navigation of the mobile robot 12, e.g. to avoid obstacles. The map is also used for controlling the pathogen detector 30 and the inactivation device 34 as described herein.

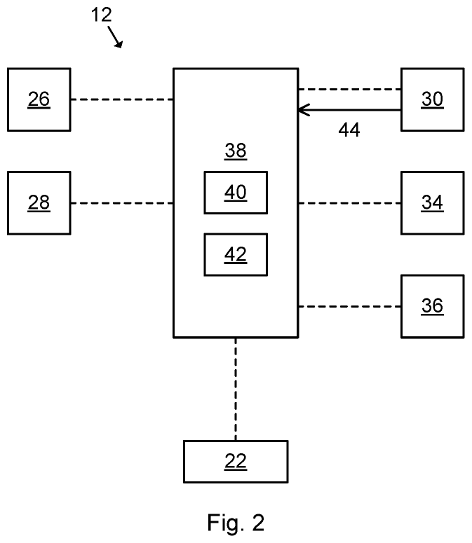

FIG. 2 schematically represents components of the mobile robot 12. As shown, the mobile robot 12 further comprises a control system 38. The control system 38 comprises a data processing device 40 and a memory 42. The memory 42 has a computer program stored thereon. The computer program comprises program code which, when executed by the data processing device 40, causes the data processing device 40 to perform, or command performance of, various steps as described herein. A detection path planner for controlling movements of the pathogen detector 30, and an inactivation path planner for controlling movements of the inactivation device 34 are implemented in the control system 38.

The control system 38 may for example be physically arranged in the base 20. As shown in FIG. 2, each of the manipulator 26, the gripper 28, the traction arrangement 22, the pathogen detector 30, the inactivation device 34 and the navigation sensor 36 is in signal communication with the control system 38.

FIG. 2 shows that pathogen data 44 is communicated from the pathogen detector 30 to the control system 38. The pathogen data 44 of this example contains information indicative of a presence of pathogens 18, position information of the pathogens 18, density information of the pathogens 18 and a type of pathogens 18, in each detection region 32. The pathogen data 44 may also indicate the absence of pathogens 18.

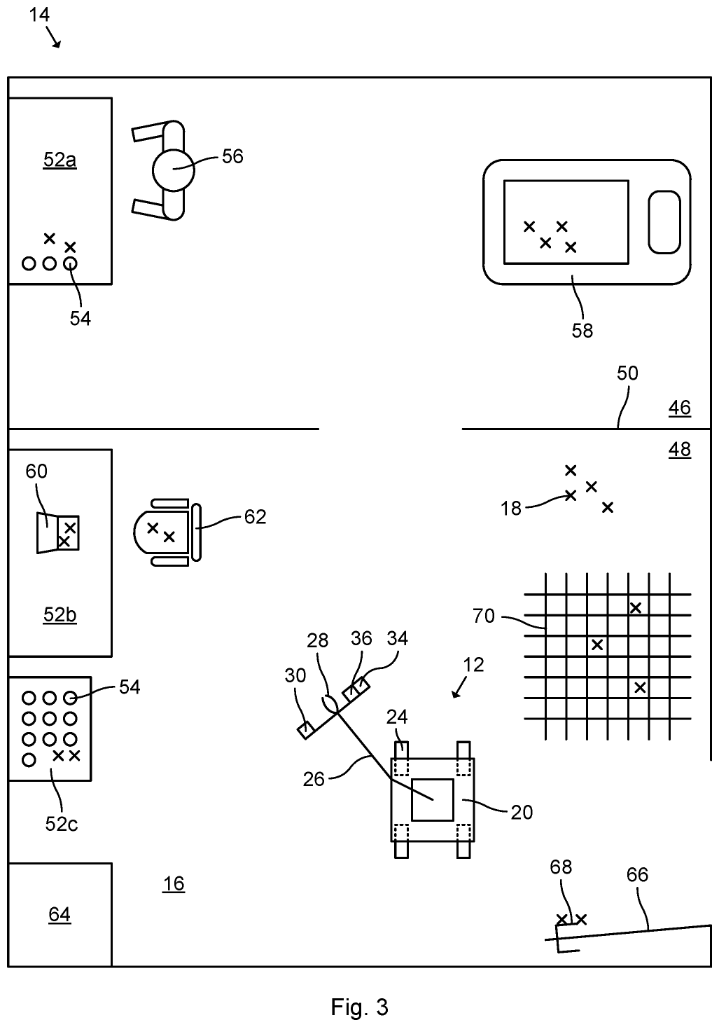

FIG. 3 schematically represents the mobile robot 12 in an environment 14 containing pathogens 18. The environment 14 of this very specific and non-limiting example is a hospital environment containing a first room 46 a second room 48 partly separated from the first room 46 by a wall 50. The first room 46 houses a first table 52a supporting items 54 such as test tubes, a human 56 and a bed 58. The second room 48 houses a second table 52b on which a laptop 60 is placed, a chair 62, a cabinet 64 and a third table 52c supporting items 54 of the same type as on the first table 52a. The mobile robot 12 may for example be assigned to carry items 54 between the first table 52a and the third table 52c by means of the gripper 28. FIG. 3 further shows a door 66 having a door handle 68.

As shown in FIG. 3, pathogens 18 are present at a wide range of locations within the environment 14 as shown by crosses. One exemplary method of inactivating these pathogens 18 by the mobile robot 12 will now be described.

FIG. 3 shows a map 70, here exemplified as a three-dimensional grid-based map representing the environment 14. The map 70 is stored in the control system 38 and may be provided by means of the navigation sensor 36.

The mobile robot 12 is autonomously controlled to execute a detection strategy using the detection path planner. The detection strategy may for example be executed once every hour. During execution of the detection strategy, the base 20 and the manipulator 26 are controlled to move such that the pathogen detector 30 scans the environment 14, such as surfaces, edges and contours thereof. Optionally, the detection strategy may be interrupted for execution of an inactivation strategy, described below.

Due to the manipulator 26, the mobile robot 12 can easily move the pathogen detector 30 for example under the tables 52a-52c, under the bed 58, on top of the cabinet 64, to the laptop 60 and to the door handle 68. Although the manipulator 26 can move the pathogen detector 30 to very large heights, e.g. more than 3 meters, scanning may optionally be limited to be performed below a certain height, e.g. to exclude the ceiling.

During the scanning, the pathogen data 44 generated by the pathogen detector 30 is continuously or successively added to the map 70. When pathogens 18 are detected, a type of pathogen 18 is determined and is included in the pathogen data 44. The locations and densities of pathogens 18 are also communicated in the pathogen data 44 from the pathogen detector 30 and are stored in association with the map 70.

The map 70 thereby provides a detailed distribution of the pathogens 18 within the environment 14 in three dimensions. For each scan for pathogens 18, the resulting pathogen data 44 may be associated with one or more cells of the grid-based map 70. In this way, several or all cells of the map 70 can be associated with pathogen data 44. In case the pathogen data 44 indicates a level of pathogens 18 above a threshold value, an alarm may be issued by the mobile robot 12.

The mobile robot 12 may immediately act to disinfect the detected pathogens 18 by means of the inactivation device 34. This mode of operation may be referred to as a patrol mode. Alternatively, the mobile robot 12 may proceed with scanning the entire environment 14 for mapping the pathogens 18 to the map 70 before proceeding with inactivating the pathogens 18. In any case, the mobile robot 12 can locally address detected pathogens 18 and deactivate the same. This generates substantial time savings and costs savings. Moreover, the overall disinfection performance of the mobile robot 12 is improved.

When the execution of the detection strategy is completed, or when the execution of the detection strategy is interrupted (e.g. due to detected pathogens 18 or due to detection of a density of pathogens 18 above a threshold value), execution of the inactivation strategy takes place. The mobile robot 12 is then autonomously controlled to execute the inactivation strategy using the inactivation path planner. During execution of the inactivation strategy, the base 20 and the manipulator 26 are controlled to move the inactivation device 34 based on the pathogen data 44 in the map 70. That is, rather than moving the inactivation device 34 over the entire environment 14, the inactivation device 34 is moved to areas where pathogens 18 have actually been detected. Similar to the movements of the pathogen detector 30 in the detection strategy, the inactivation device 34 can easily and accurately reach the areas where inactivation of pathogens 18 is needed. The entire environment 14 can thereby be efficiently disinfected. Once execution of the inactivation strategy is completed, the mobile robot 12 can return to perform other tasks or may be placed at rest. The detection strategy may also be executed simultaneously with the mobile robot 12 moving to perform other tasks, such as transportation tasks.

The detection strategy and the inactivation strategy may be continuously improved by machine learning algorithms implemented in the control system 38. For example, the detection strategy can be adapted to start scanning for pathogens 18 in areas where pathogens 18 are often found. If pathogens 18 occur at the same locations several times, the inactivation strategy may be executed several times between two executions of the detection strategy. As a further example, the inactivation strategy can try to enhance the inactivation at areas where pathogens 18 are often found. If the occurrence of pathogens 18 can be reduced (as determined by execution of a subsequent detection strategy) in areas where pathogens 18 often occur by such enhanced inactivation, the inactivation strategy can be improved accordingly.

Execution of the detection strategy and the inactivation strategy may be prioritized over the normal tasks (here moving items 54) or vice versa. Thus, when the inactivation of pathogens 18 within the environment 14 is considered sufficient, the mobile robot 12 may return to execution of its normal tasks. Since the mobile robot 12 is collaborative, and by using an inactivation device 34 not harmful to humans, the human 56 can continue to perform her/his tasks during execution of the detection strategy and the inactivation strategy by the mobile robot 12.

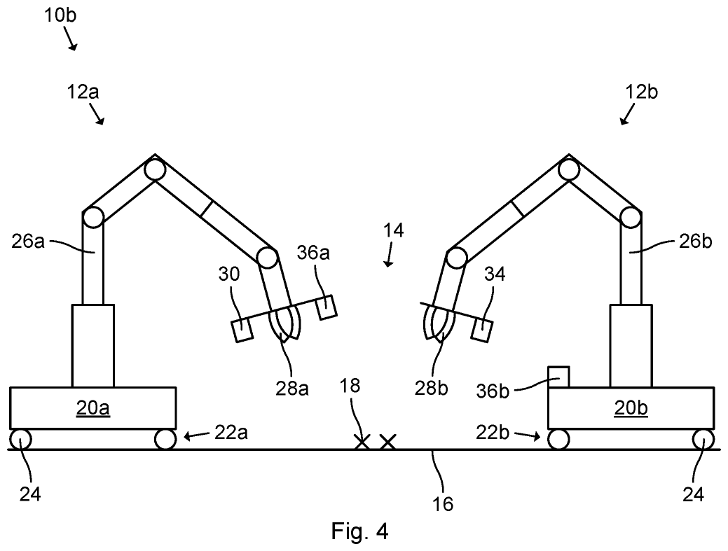

FIG. 4 schematically represents a side view of a further example of a robot system 10b. Differences with respect to the robot system 10b will be described. The robot system 10b comprises a first mobile robot 12a and a second mobile robot 12b. The first mobile robot 12a comprises a first traction arrangement 22a, a first base 20a, a first manipulator 26a, a first end effector 28a and a first navigation sensor 36a. The second mobile robot 12b comprises a second traction arrangement 22b, a second base 20b, a second manipulator 26b, a second end effector 28b and a second navigation sensor 36b. The traction arrangements 22a and 22b are of the same type as the traction arrangement 22. The bases 20a and 20b are of the same type as the base 20. The manipulators 26a and 26b are of the same type as the manipulator 26. The end effectors 28a and 28b are of the same type as the end effector 28.

The navigation sensors 36a and 36b are of the same type as the navigation sensor 36. However, the second navigation sensor 36b is positioned on the second base 20b instead of being carried by the second manipulator 26b. The first navigation sensor 36a is carried by the first manipulator 26a.

The first manipulator 26a carries the pathogen detector 30. The second manipulator 26b carries the inactivation device 34. Thus, with the robot system 10b, the detection strategy may be performed by the first mobile robot 12a and the inactivation strategy may be performed by the second mobile robot 12b.

While the present disclosure has been described with reference to exemplary embodiments, it will be appreciated that the present invention is not limited to what has been described above. For example, it will be appreciated that the dimensions of the parts may be varied as needed. Accordingly, it is intended that the present invention may be limited only by the scope of the claims appended hereto.

1. A method of inactivating pathogens in an environment by means of a robot system, the robot system comprising:
at least one mobile robot, each mobile robot having a base, a traction arrangement configured to move the base on a base surface of the environment, and at least one manipulator connected to the base and movable relative to the base;
a pathogen detector carried by one of the at least one manipulator, the pathogen detector being configured to detect the presence of pathogens in a detection region; and
an inactivation device carried by one of the at least one manipulator, the inactivation device being configured to inactivate pathogens; the method including the steps of:
moving the base on the base surface;
moving the pathogen detector by means of the manipulator;
obtaining pathogen data by means of the pathogen detector, the pathogen data being indicative of a presence of pathogens in one or more detection regions of the environment;
moving the base on the base surface based on the pathogen data;
moving the inactivation device based on the pathogen data by means of the manipulator; and
controlling the inactivation device to inactivate the pathogens.

2. The method according to claim 1, wherein each base and each manipulator is moved autonomously.

3. The method according to claim 1, further comprising:
providing a maps representing the environment; and
mapping the pathogen data in the map.

4. The method according to claim 3, wherein the moving and controlling of the inactivation device is made based on the map.

5. The method according to claim 3, further comprising providing a detection strategy based on the map, wherein the moving of the pathogen detector is performed based on the detection strategy.

6. The method according to claim 3, further comprising providing an inactivation strategy based on the map, wherein the moving and controlling of the inactivation device is performed based on the inactivation strategy.

7. The method according to claim 3, wherein the map is three-dimensional.

8. The method according to claim 1, further comprising issuing an alarm if the pathogen data indicates a level of pathogens in a detection region above a threshold value.

9. The method according to claim 1, wherein one of the at least one manipulator comprises an end effector.

10. The method according to claim 1, wherein each mobile robot is a collaborative robot.

11. The method according to claim 1, wherein the pathogen detector and the inactivation device are noninvasive.

12. The method according to claim 1, wherein the pathogen detector and the inactivation device are provided on a common manipulator.

13. A control system for controlling inactivation of pathogens in an environment by means of a robot system, the robot system comprising:
at least one mobile robot, each mobile robot having a base, a traction arrangement configured to move the base on a base surface of the environment, and at least one manipulator connected to the base and movable relative to the base;
a pathogen detector carried by one of the at least one manipulator, the pathogen detector being configured to detect the presence of pathogens in a detection region; and
an inactivation device carried by one of the at least one manipulator, the inactivation device being configured to inactivate pathogens; the control system having at least one data processing device and at least one memory having at least one computer program stored thereon, the at least one computer program including program code which, when executed by the at least one data processing device, causes the at least one data processing device to perform the steps of:
commanding movement of the base carrying the pathogen detector on the base surface;
commanding movement of the pathogen detector by means of the manipulator carrying the pathogen detector;
receiving pathogen data from the pathogen detector, the pathogen data being indicative of a presence of pathogens in one or more detection regions of the environment;
commanding movement of the base carrying the inactivation device on the base surface based on the pathogen data;
commanding movement of the inactivation device based on the pathogen data by means of the manipulator carrying the inactivation device; and
controlling the inactivation device to inactivate the pathogens.

14. The control system according to claim 13, wherein the at least one computer program comprises program code which, when executed by the at least one data processing device, causes the at least one data processing device to perform, or command performance of, the method according to any of claims 2 to 12.

15. A robot system comprising:
the at least one mobile robot;
the pathogen detector;
the inactivation device; and
a control system for controlling inactivation of pathogens in an environment, the robot system comprising:
at least one mobile robot, each mobile robot having a base, a traction arrangement configured to move the base on a base surface of the environment and at least one manipulator connected to the base and movable relative to the base,
a pathogen detector carried by one of the at least one manipulator, the pathogen detector being configured to detect the presence of pathogens in a detection region; and
an inactivation device carried by one of the at least one manipulator, the inactivation device being configured to inactivate pathogens;
the control system having at least one data processing device and at least one memory having at least one computer program stored thereon, the at least one computer program including program code which, when executed by the at least one data processing device, causes the at least one data processing device to perform the steps of:
commanding movement of the base carrying the pathogen detector on the base surface;
commanding movement of the pathogen detector by means of the manipulator carrying the pathogen detector;
receiving pathogen data from the pathogen detector, the pathogen data being indicative of a presence of pathogens in one or more detection regions of the environment;
commanding movement of the base carrying the inactivation device on the base surface based on the pathogen data;
commanding movement of the inactivation device based on the pathogen data by means of the manipulator carrying the inactivation device; and
controlling the inactivation device to inactivate the pathogens.

16. The method according to claim 2, further comprising:
providing a map representing the environment; and
mapping the pathogen data in the map.

17. The method according to claim 2, further comprising issuing an alarm if the pathogen data indicates a level of pathogens in a detection region above a threshold value.

18. The method according to claim 4, further comprising providing a detection strategy based on the map, wherein the moving of the pathogen detector is performed based on the detection strategy.

19. The method according to claim 4, further comprising providing an inactivation strategy based on the map, wherein the moving and controlling of the inactivation device is performed based on the inactivation strategy.

20. The method according to claim 4, wherein the map is three-dimensional.
